(19) United States
(12) Patent Application Publication
(10) Pub. No.: US 2010/0006098 A1
(43) Pub. Date: Jan. 14, 2010

(54) CPAP-OXIMETER HYBRID DEVICE AND METHOD OF USING

(76) Inventors: William J. McGinnis, Cincinnati, OH (US); Scott A. Metrick, Glenview, IL (US)

Correspondence Address: LADAS & PARRY LLP, 224 SOUTH MICHIGAN AVENUE, SUITE 1600, CHICAGO, IL, 60604, US

(21) Appl. No.: 12/170,516

(22) Filed: Jul. 10, 2008

(51) Int. Cl. A61M 16/00

(52) U.S. Cl. 128/204.23

(57) ABSTRACT

A CPAP-oximeter hybrid device and an associated method of using are presented for use in automatically adjusting or automatically controlling an air ventilation flow rate as a function of an estimated oxygen level in a patient using the device. The CPAP-oximeter hybrid device includes the interconnected components of a mouthpiece; a first and second EMF emitter; a detector; a housing unit, a blower and a control circuit. The method of using the CPAP-oximeter hybrid device includes the steps of adjusting, allowing, inserting, obtaining, reviewing, removing, setting, switching, and transferring.

### FIELD OF THE INVENTION

The present invention relates to treatment and/or diagnosis of partial or complete upper airway occlusion. More particularly to a continuous pressure airwave pressure (CPAP)-oximeter hybrid device configured to provide a variable CPAP flow rate as a function of estimated oxygen saturation levels in the patient.

### BACKGROUND OF THE INVENTION

Sleep apnea is a syndrome where a person stops breathing during sleep. When the airflow ceases for more than 10 seconds it is called “apnea”. Apneas can lead to decreased blood oxygenation and can often disrupt sleep. Apneas can be categorized as either central apneas, where there is no respiratory effort, or as obstructive apneas, where there is respiratory effort. In some central apneas and all obstructive apneas, the airway becomes completely closed. This closure usually occurs at the level of the tongue or soft palate. The airway may also be only partially obstructed which can also lead to decreased ventilation (hypopnea) and decreased blood oxygenation, as well as, disturbed sleep.

Treatment to minimize or eliminate these sleep apnea events is important because sleep apnea can aggravate and even induce more serious clinical conditions, e.g., cardiovascular disease and stroke. Accordingly, patients that suffer from sleep apnea may significantly benefit from the therapy of using positive pressure ventilator support while these patients sleep.

A well-known therapy for sleep apnea is the administration of Continuous Positive Airway Pressure (CPAP). It is thought that the CPAP treatment acts as a pneumatic splint of the airway by the provision of a positive pressure. The ancillary air is often supplied to the airway by a motor driven blower whose outlet passes via an air delivery hose to a nose (or nose and/or mouth) mask that is sealingly engaged to a patient's face. An exhaust port is often provided somewhere along the delivery tube proximate to the mask. The mask is often either a nose and/or face mask or nasal prongs, pillows or cannulae.

Unfortunately basic CPAP ventilators do not provide real-time monitoring of the patients. Accordingly, basic CPAP ventilators do not provide variable flow rates of the CPAP ventilation as a function of oxygen saturation levels in blood and tissue. In general, patients benefit from using a basic CPAP ventilator therapy, however many patients can benefit even more if the CPAP ventilator could proportionately provide flow rates of air as a real-time function of blood and/or tissue oxygen levels.

While presently known CPAP techniques fulfill many of their respective objectives and requirements, no CPAP techniques or devices describe a CPAP-oximeter hybrid device having the interconnected components of a mouthpiece; a first EMF emitter; a second EMF; a detector; a housing unit; a blower; and a control circuit for real-time flow-rate control as a function of the patient's oxygen levels and/or for diagnosing sleep apnea disorders. This combination of interconnected elements would specifically match the user's particular individual needs of making it possible to provide a means for treating and/or diagnosing sleep apnea disorders in a convenient manner.

Therefore, a need exists for a new and improved CPAP device and an associated method of using this new and improved CPAP-oximeter hybrid device for treating and/or diagnosing sleep apnea. In this respect, the CPAP-oximeter hybrid device according to the present invention substantially departs from the conventional concepts and designs of the prior art, and in doing so provides an apparatus primarily developed for the purpose of providing a convenient means for making it possible to treat and/or diagnose sleep apnea.

### SUMMARY OF THE INVENTION

The present device and method of using, according to the principles of the present invention, overcomes a number of the shortcomings of the prior art by providing a novel CPAP-oximeter hybrid device and method for use in treating patients suffering from sleep apnea. The CPAP-oximeter hybrid device includes the interconnected components of a mouthpiece; a first EMF emitter; a second EMF; a detector; a housing unit; a blower; and a control circuit where the control circuit is configured to adjust a flow rate of the blower into the ventilator passageway as a function of a detected signal from the detector. The method includes the steps of adjusting, allowing, inserting, obtaining, reviewing, removing, setting, switching, and transferring.

In view of the foregoing disadvantages inherent in the known type CPAP devices now present in the prior art, the present invention provides an improved CPAP-oximeter hybrid device, which will be described subsequently in great detail, is to provide a new and improved CPAP-oximeter hybrid device which is not anticipated, rendered obvious, suggested, or even implied by the prior art, either alone or in any combination thereof.

To attain this, the present invention essentially comprises the interconnected components of a mouthpiece; a first EMF emitter; a second EMF; a detector; a housing unit; a blower; and a control circuit where the control circuit is configured to adjust a flow rate of the blower into the ventilator passageway as a function of a detected signal from the detector.

There has thus been outlined, rather broadly, the more important features of the invention in order that the detailed description thereof that follows may be better understood, and in order that the present contribution of the art may be better appreciated.

Numerous aspects, features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an aspect of the present invention to provide a new and improved CPAP device that has many of the advantages of the prior CPAP device and minimizing a number of their disadvantages.

It is another aspect of the present invention to provide a new and improved CPAP device that may be easily and efficiently manufactured and marketed.

An even further aspect of the present invention is to provide a new and improved CPAP device that has a low cost of manufacture with regard to both materials and labor, and which accordingly is then susceptible of low prices of sale to the consuming public, thereby making the CPAP-oximeter hybrid device economically available to the buying public.

Still another aspect of the present invention is to provide a CPAP-oximeter hybrid device that provides some of the advantages over basic CPAP devices, while simultaneously overcoming some of the disadvantages normally associated therewith.

Even still another aspect of the present invention is to provide a CPAP-oximeter hybrid device having the interconnected components of a mouthpiece, a first EMF emitter; a second EMF; a detector; a housing unit; a blower; and a control circuit where the control circuit is configured to adjust a flow rate of the blower into the ventilator passageway as a function of a detected signal from the detector.

Lastly, it is an aspect of the present invention to provide a new and improved method of using comprising the steps of adjusting, allowing, inserting, obtaining, reviewing, removing, setting, switching, and transferring.

There has thus been outlined, rather broadly, the more important features of the invention in order that the detailed description thereof that follows may be better understood, and in order that the present contribution of the art may be better appreciated.

Numerous other features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other aspects of the invention, along with the various features of novelty that characterize the invention, are pointed out with particularity in the claims annexed to and forming a part of this disclosure. For a better understanding of the invention, its operating advantages and the specific aspects attained by its uses, reference should be had to the accompanying drawings and description matter in which there are illustrated preferred embodiments of the invention.

### BRIEF DESCRIPTION OF THE DRAWINGS

The invention will be better understood and aspects other than those set forth above will become apparent when consideration is given to the following detailed description thereof. Such description makes reference to the annexed drawings wherein:

FIG. 1 is a perspective view of an embodiment of the CPAP-oximeter hybrid device constructed in accordance with the principles of the present invention;

FIG. 2 is a cross-sectional view of a transmittance embodiment CPAP-oximeter hybrid device of the present invention;

FIGS. 3A and 3B are partial cross-sectional views of two different embodiments of CPAP-oximeter hybrid device of the present invention;

FIG. 4 is a circuit block diagram of the control circuit of an embodiment of the CPAP-oximeter hybrid device of the present invention; and

FIG. 5 is a logical block diagram of some of the operational procedures implemented by the CPAP-oximeter hybrid device of the present invention. The same reference numerals refer to the same parts throughout the various figures.

### DETAILED DESCRIPTION OF THE PREFERRED EMBODIMENT

The following detailed embodiments presented herein are for illustrative purposes. That is, these detailed embodiments are intended to be exemplary of the present invention for the purposes of providing and aiding a person skilled in the pertinent art to readily understand how to make and use of the present invention.

Accordingly, the detailed discussion herein of one or more embodiments is not intended, nor is to be construed, to limit the metes and bounds of the patent protection afforded the present invention, in which the scope of patent protection is intended to be defined by the claims and their equivalents thereof. Therefore, embodiments not specifically addressed herein, such as adaptations, variations, modifications, and equivalent arrangements, should be and are considered to be implicitly disclosed by the illustrative embodiments and claims described herein and therefore fall within the scope of the present invention.

Further, it should be understood that, although steps of various the claimed method may be shown and described as being in a sequence or temporal order, the steps of any such method are not limited to being carried out in any particular sequence or order, absent an indication otherwise. That is, the claimed method steps are to be considered to be capable of being carried out in any sequential combination or permutation order while still falling within the scope of the present invention.

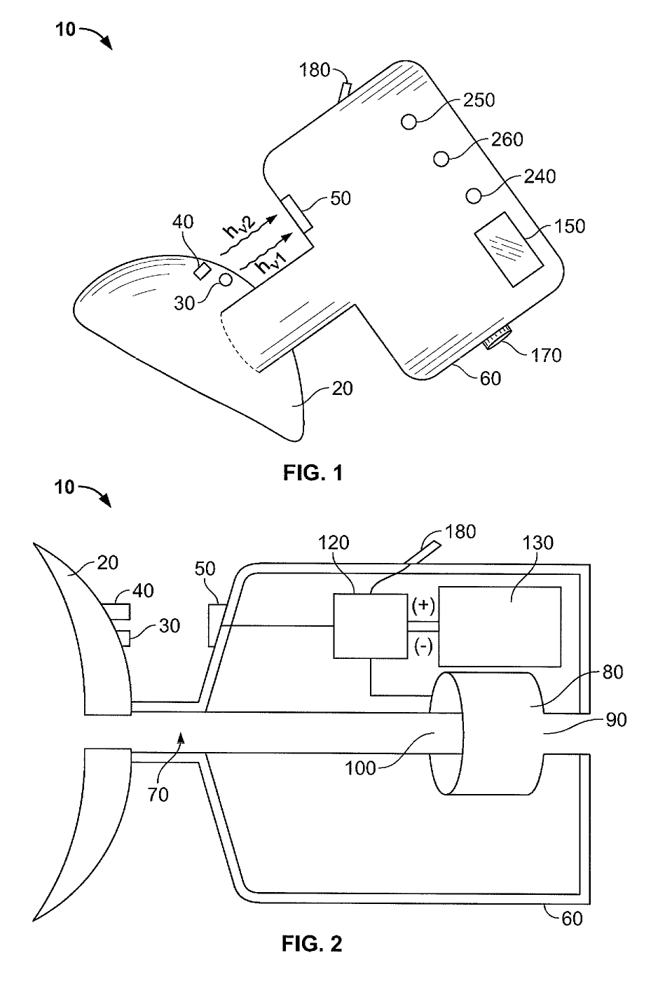

Referring now to the drawings, and in particular FIGS. 1 to 5 thereof, one preferred embodiment of the present invention is shown and generally designated by the reference numeral 10. One preferred embodiment of the continuous positive airway pressure (CPAP)-oximeter hybrid device 10 comprises a mouthpiece 20; a first EMF emitter 30; a second EMF emitter 40; a detector 50; a housing unit 60; a blower 80; and a control circuit 110. The first EMF emitter 30 is attached to the mouthpiece 20 in which the first EMF emitter 30 is configured to emit a first EMF emission. The second EMF emitter 40 is attached to the mouthpiece 20 in which the second EMF emitter 40 is configured to emit a second EMF emission. The detector 50 is configured to receive the first and second EMF emissions. The housing unit 60 is attached to the mouthpiece 20 in which the housing unit 60 and the mouthpiece 20 commonly share a ventilator passageway 70. The blower 80 is attached to the housing unit 60 in which the blower 80 has an inlet 90 and an outlet 100 wherein the inlet 90 and the outlet 100 of the blower 80 are in fluid communications with the ventilator passageway 70. The control circuit 110 is electrically coupled to the first and second EMF emitter (30 and 40, respectively), to the detector 50, and to the blower 80. The control circuit 110 is configured to adjust a flow rate of the blower 80 into the ventilator passageway 70 as a function of a detected signal from the detector 50.

The first and second EMF emitter (30 and 40, respectively) may be any type of known EMF emitters such as being light emitting diodes (LEDs) or lasers. Accordingly, the first and second EMF emissions may be either emit broadband EMF emissions or monochromatic EMF emissions. One preferred embodiment of the first EMF emitter 30 is that it is configured to emit the first EMF emission having a red emission between about 625 to about 740 nanometers. A more preferred embodiment is that the first EMF emitter 30 is configured to emit the first EMF emission that has a mean red emission frequency at about 660 nanometers. One preferred embodiment of the second EMF emitter 40 is that it is configured to emit the second EMF emission between about 800 to about 1100 nanometers. A more preferred embodiment of the second EMF emitter 40 is configured to emit the second EMF emission having a mean infrared emission frequency at about 940 nanometers.

The detector 50 may be any known type of photodetector 50 such as those selected from the group consisting of photodetectors, photodiodes, pin diodes, phototransistors, a charge-coupled device 10 (CCD) array, and photomultiplier tube. Further to reduce extraneous unwanted light, the detector 50 may have a spectral bandpass filter for selectively enhancing the signal to noise sensitivity. The detector 50 may be mounted anywhere on the CPAP-oximeter hybrid device 10 so long as, it is able to somehow be able to be optically coupled to the first and second EMF emitters (30 and 40, respectively). One preferred embodiment is that the detector 50 is attached to the housing unit 60 so that detector 50 is configured to detect the first and second EMF emissions transmitted through tissue in accordance to a transmittance oximetry measurement scheme. Another preferred embodiment is that the detector 50 is attached to the mouthpiece 20 so that detector 50 is configured to detect the first and second EMF emissions scattered by tissue in accordance to a reflectance oximetry measurement scheme.

The control circuit 110 may have any number of different configurations that can perform any number of different tasks. One preferred configuration is that the control circuit 110 is configured to adjust the blower 80 flow rate in response to a detector 50 signal used to estimate an oxygen saturation value in tissue. Another preferred configuration is that the control circuit 110 is configured to control the first and second EMF emitter (30 and 40, respectively) in an pulse mode. Yet another preferred configuration is that the control circuit 110 comprises an alarm unit 240 configured to emit an audible pulse as a function of the detected signal from the detector 50 falling below a threshold limit. The control circuit 110 may a visual display 150 for displaying various modes such as an on/off operating mode, a manual threshold setting mode, a manual flow rate setting mode, an alarm mode, a data transfer mode. Accordingly, the control circuit 110 may be comprise a control unit 120, a power source 130, a clock 140, a visual display 150, a memory buffer 160, a data output port 170, an ON/OFF switch 180, an A/D converter 190, a first sampler 200, a second sampler 210, an amplifier 220, an EMF drive unit 230, an alarm unit 240, a flow rate set unit 250 and a threshold set unit 260. Components of the control circuit 110 may be attached at any number of different locations along the CPAP-oximeter hybrid device 10 such as having some of the control circuit 110 attached to the mouthpiece 20.

One preferred embodiment of a method of using the CPAP-oximeter hybrid device 10 for providing a variable CPAP therapy that responds to a real time function of an estimated oxygen saturation value in a patient, the method comprising the steps of: adjusting, allowing, inserting, obtaining, reviewing, removing, setting, switching, and transferring. The obtaining step comprises obtaining the CPAP-oximeter hybrid device 10 comprising: a mouthpiece 20; a first EMF emitter 30 attached to the mouthpiece 20, wherein the first EMF emitter 30 is configured to emit a first EMF emission; a second EMF emitter 40 is attached to the mouthpiece 20, wherein the second EMF emitter 40 is configured to emit at a second EMF emission; a detector 50 is configured to receive the first and second EMF emissions wherein the detector 50 is attached to the housing unit 60 so that detector 50 is configured to detect the first and second EMF emissions transmitted through tissue in accordance to a transmittance oximetry measurement scheme; a housing unit 60 is attached to the mouthpiece 20, wherein the housing unit 60 and the mouthpiece 20 commonly share a ventilator passageway 70; a blower 80 is attached to the housing unit 60, wherein the blower 80 has an inlet 90 and an outlet 100, in which the inlet 90 and the outlet 100 of the blower 80 are in fluid communications with the ventilator passageway 70; and a control circuit 110 electrically coupled to the first and second EMF emitter (30 and 40, respectively), to the detector 50, and to the blower 80, wherein the control circuit 110 is configured to adjust a flow rate of the blower 80 into the ventilator passageway 70 as a function of a detected signal from the detector 50, wherein the control circuit 110 is configured to adjust the blower 80 flow rate in response to a detector 50 signal used to estimate an oxygen saturation value in tissue, wherein the control circuit 110 the control circuit 110 comprises a control unit 120, a power source 130, a clock 140, a visual display 150, a memory buffer 160, a data output port 170, an ON/OFF switch 180, an A/D converter 190, a first sampler 200, a second sampler 210, an amplifier 220, an EMF drive unit 230, an alarm unit 240, a flow rate set unit 250 and a threshold set unit 260. The inserting step comprises inserting the mouthpiece 20 of the CPAP-oximeter hybrid device 10 into a mouth of the patient. The switching step comprises switching on the ON/OFF switch 180 to turn on the CPAP-oximeter hybrid device 10. The adjusting step comprises adjusting manually the flow rate of the blower 80 using the flow rate set unit 250. The setting step comprises setting a minimum oxygen threshold limit using the threshold set unit 260. The allowing step comprises allowing the patient to sleep with the CPAP-oximeter hybrid device 10 secured in the mouth of the patient. The removing step comprises removing the mouthpiece 20 of the CPAP-oximeter hybrid device 10 from the mouth of the patient after the patient awakens. The transferring step comprises transferring information stored in the memory buffer 160 through the data output port 170. The reviewing step comprises reviewing the transferred information to aid in a sleep apnea diagnosis of the patient.

Referring now to FIG. 1 that depicts a perspective view of an embodiment of the CPAP-oximeter hybrid device 10 showing the mouthpiece 20; the first EMF emitter 30; the second EMF emitter 40; the detector 50; and the housing unit 60. Also shown are the visual display 150, the data output port 170, the ON/OFF switch 180, the alarm unit 240, the flow rate set unit 250 and the threshold set unit 260 which are constituents of the control circuit 110. Because the first and second EMF emitters 30,40 are configured to be opposed to the detector 50, this depicted CPAP-oximeter hybrid device 10 is intended to function in a transmittance oximeter operational mode.

Referring now to FIG. 2 that depicts a cross-sectional view of a CPAP-oximeter hybrid device 10 showing a mouthpiece 20; the first EMF emitter 30; the second EMF emitter 40; the detector 50; the housing unit 60; and the blower 80. The blower 80 is shown having an inlet 90 and an outlet 100. The ventilator passageway 70 is shown commonly shared between the housing unit 60 and the mouthpiece 20. Also shown are the control unit 120, the power source 130, the ON/OFF switch 180 of the control circuit 110. Note that because the first and second EMF emitters 30,40 are configured to be opposed to the detector 50, then depicted CPAP-oximeter hybrid device 10 is intended to function in a transmittance oximeter operational mode.

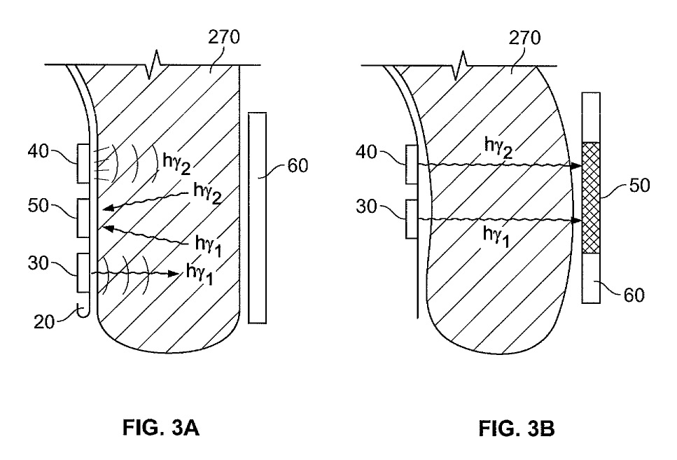

Referring now to FIG. 3A depicts a partial cross-sectional view of one embodiment of the CPAP-oximeter hybrid device 10 that is intended to function in a reflectance oximeter operational mode. In particular FIG. 3A shows that the first and second EMF emitters 30,40 and the detector 50 are not configured to be opposed to each other. Radiant energy is shown emitted from the first and second EMF emitters 30,40 into an adjacently placed tissue 270, e.g., a lip of a patient. This emitted energy is scattered throughout the patient's tissue 270 in which some of this scattered emitted energy is back scattered towards the detector 50. Accordingly the detector 50 detects a portion of this back scattered emitted energy and consequently the detector 50 signal can be used to estimate the blood oxygen level in the patient's tissue 270.

Referring now to FIG. 3B depicts a partial cross-sectional views of another embodiment of the CPAP-oximeter hybrid device 10 that is intended to function in a transmittance oximeter operational mode. In particular FIG. 3B shows that the first and second EMF emitters 30,40 and the detector 50 are configured to be opposed to each other. Radiant energy is shown emitted from the first and second EMF emitters 30,40 and into an adjacently placed tissue 270, e.g., a lip of a patient. This radiant energy is shown traversing entirely through the adjacently placed tissue 270 and directly arriving at the detector 50. Accordingly the detector 50 detects a portion of this transmitted emitted energy and consequently the detector 50 signal can be used to estimate the blood oxygen level in the patient's tissue 270.

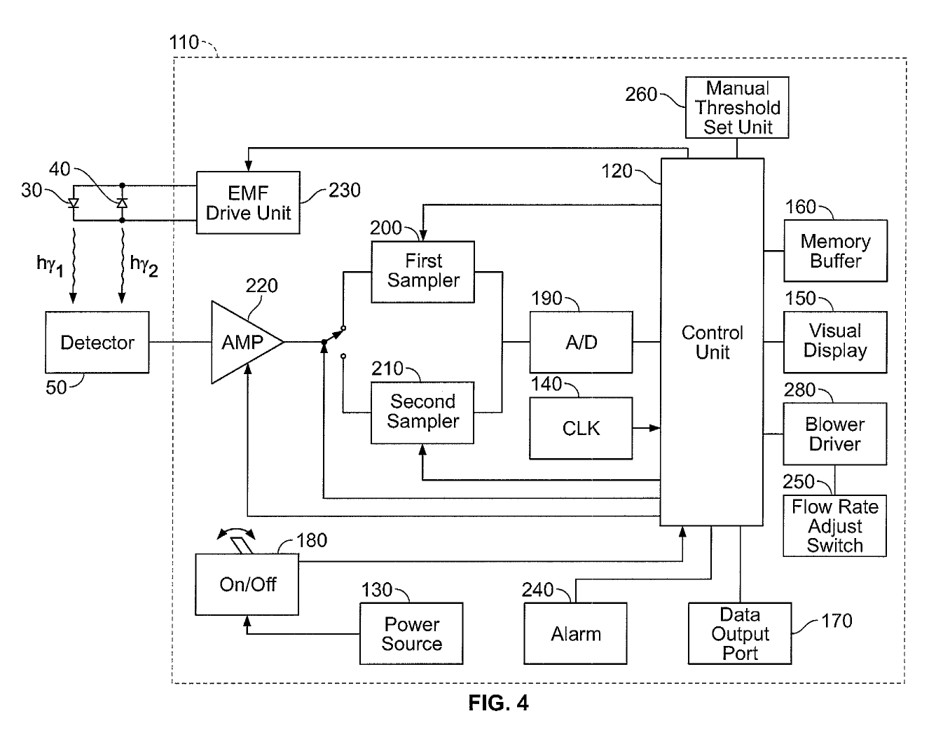

Referring now to FIG. 4 that depicts a circuit block diagram of an embodiment of the control circuit 110 of an embodiment of the CPAP-oximeter hybrid device 10. The control circuit 110 is shown having the interconnected components of the control unit 120, the power source 130, the clock 140, the visual display 150, the memory buffer 160, the data output port 170, the ON/OFF switch 180, the A/D converter 190, the first sampler 200, the second sampler 210, the amplifier 220, the EMF drive unit 230, the alarm unit 240, the flow rate set unit 250, the blower driver 280, and the threshold set unit 260. Also shown is the detector 50 optically coupled to the first and second EMF emitters 30, 40 and electrically coupled to the amplifier 220 of the control circuit 110. Also shown is the first and second EMF emitters 30, 40 are electrically coupled to the EMF drive unit 230 of the control circuit 110.

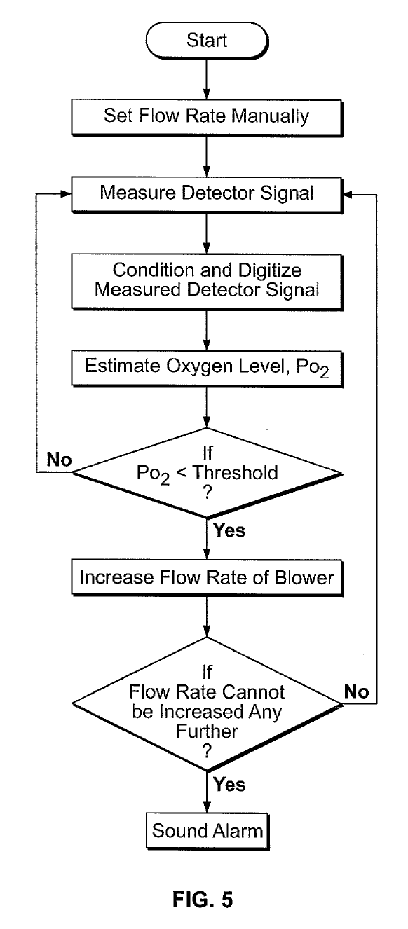

Referring now to FIG. 5 that depicts a logical block diagram of some of the operational procedures implemented by the CPAP-oximeter hybrid device 10 of the present invention. The CPAP-oximeter hybrid device 10 may have an optional the flow rate set unit 250 for manually setting the initial flow rate of the CPAP-oximeter hybrid device 10. When the CPAP-oximeter hybrid device 10 is functioning the detector 50 can produce a signal in response to a detected portion of the emitted radiation from the first and second EMF emitters 30, 40 so that the control circuit 110 can eventually estimate the blood oxygen level in the patients tissue 270. The control circuit 110 may optionally condition and digitize the measured detector signal. In the event that the blood oxygen level in the patient's tissue 270 is above the set threshold, then the CPAP-oximeter hybrid device 10 simply repeats a cycle of measuring the detector signal. In the event that the blood oxygen level in the patients tissue 270 falls below the set threshold, then the CPAP-oximeter hybrid device 10 increases the flow rate of the blower 80 to provide more air to the patient. In the event that the flow rate cannot be further increased then the CPAP-oximeter hybrid device 10 enables the alarm unit 240 to produce an audible signal for indicating that the patient may be in distress.

As to the manner of usage and operation of the present invention, the same should be apparent from the above description. Accordingly, no further discussion relating to the manner of usage and operation will be provided.

While preferred embodiments of the CPAP-oximeter hybrid device and method of using same has been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Those skilled in the art will appreciate that the invention described herein is susceptible to variations and modifications other than those specifically described. It is to be understood that the invention includes all such variations and modifications that fall within its spirit and scope. The invention also includes all of the steps, features, compositions and compounds referred to or indicated in this specification, individually or collectively, and any and all combinations of any two or more of said steps or features.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. A continuous positive airway pressure (CPAP)-oximeter hybrid device comprising:
a mouthpiece;
a first electromagnetic force (EMF) emitter attached to the mouthpiece wherein the first EMF emitter is configured to emit a first EMF emission;
a second EMF emitter attached to the mouthpiece wherein the second EMF emitter is configured to emit a second EMF emission;
a detector configured to receive the first and second EMF emissions;
a housing unit attached to the mouthpiece, wherein the housing unit and the mouthpiece having a commonly shared ventilator passageway;
a blower attached to the housing unit, the blower having an inlet and an outlet, the inlet and the outlet of the blower in fluid communications with the ventilator passageway; and
a control circuit electrically coupled to the first and second EMF emitters, to the detector, and to the blower, wherein the control circuit is configured to adjust a flow rate of the blower into the ventilator passageway as a function of a detected signal from the detector.

2. The device of claim 1 wherein the detector is attached to the housing unit so that detector is configured to detect the first and second EMF emissions transmitted through tissue in accordance to a transmittance oximetry measurement scheme.

3. The device of claim 1 wherein the detector is attached to the mouthpiece so that detector is configured to detect the first and second EMF emissions scattered by tissue in accordance to a reflectance oximetry measurement scheme.

4. The device of claim 1 wherein the control circuit is configured to adjust the blower flow rate in response to a detector signal used to estimate an oxygen saturation value in tissue.

5. The device of claim 1 wherein the first EMF emitter is configured to emit the first EMF emission having a red emission between about 625 to about 740 nanometers.

6. The device of claim 5 wherein the first EMF emitter is configured to emit the first EMF emission having a mean red emission frequency at about 660 nanometers.

7. The device of claim 1 wherein the second EMF emitter is configured to emit the second EMF emission between about 800 to about 1100 nanometers.

8. The device of claim 7 wherein the second EMF emitter is configured to emit the second EMF emission having a mean infrared emission frequency at about 940 nanometers.

9. The device of claim 1 wherein the first and second EMF emitters are light emitting diodes (LEDs).

10. The device of claim 1 wherein the first and second EMF emitters are lasers.

11. The device of claim 1 wherein the first and second EMF emissions are monochromatic EMF emissions.

12. The device of claim 1 wherein the first and second EMF emissions are broadband EMF emissions.

13. The device of claim 1 wherein the detector has a spectral bandpass filter.

14. The device of claim 1 wherein the detector is selected from the group consisting of photodetectors, photodiodes, pin diodes, phototransistors, a charge-coupled device (CCD) array, and photomultiplier tube.

15. The device of claim 1 wherein the control circuit controls the first and second EMF emitters in an pulse mode.

16. The device of claim 1 wherein the control circuit comprises a control unit, a power source, a clock, a visual display, a memory buffer, a data output port, an ON/OFF switch, an A/D converter, a first sampler, a second sampler, an amplifier, an EMF drive unit, an alarm unit, a flow rate set unit and a threshold set unit.

17. The device of claim 1 wherein the control circuit further is attached to the mouthpiece.

18. The device of claim 1 wherein the control circuit comprises an alarm unit configured to emit an audible pulse as a function of the detected signal from the detector.

19. A continuous positive airway pressure (CPAP)-oximeter hybrid device comprising:
a mouthpiece;
a first electromagnetic force (EMF) emitter attached to the mouthpiece wherein the first EMF emitter is configured to emit a first EMF emission;
a second EMF emitter attached to the mouthpiece wherein the second EMF emitter is configured to emit a second EMF emission;
a detector configured to receive the first and second EMF emissions wherein the detector is attached to the housing unit so that detector is configured to detect the first and second EMF emissions transmitted through tissue in accordance to a transmittance oximetry measurement scheme;
a housing unit attached to the mouthpiece, wherein the housing unit and the mouthpiece having a commonly shared ventilator passageway;
a blower attached to the housing unit, the blower having an inlet and an outlet, the inlet and the outlet of the blower in fluid communications with the ventilator passageway; and
a control circuit electrically coupled to the first and second EMF emitters, to the detector, and to the blower,
wherein the control circuit is configured to adjust a flow rate of the blower into the ventilator passageway as a function of a detected signal from the detector,
wherein the control circuit is configured to adjust the blower flow rate in response to a detector signal used to estimate an oxygen saturation value in tissue,
wherein the control circuit the control circuit comprises a control unit, a power source, a clock, a visual display, a memory buffer, a data output port, an ON/OFF switch, an A/D converter, a first sampler, a second sampler, an amplifier, an EMF drive unit, an alarm unit, a flow rate set unit and a threshold set unit.

20. A method of using a continuous positive airway pressure (CPAP)-oximeter hybrid device for providing a variable CPAP therapy that responds to a real time function of an estimated oxygen saturation value in a patient, the method comprising the steps of:
obtaining the device comprising:
a mouthpiece;
a first electromagnetic force (EMF) emitter attached to the mouthpiece wherein the first EMF emitter is configured to emit a first EMF emission;
a second EMF emitter attached to the mouthpiece wherein the second EMF emitter is configured to emit a second EMF emission;
a detector configured to receive the first and second EMF emissions wherein the detector is attached to the housing unit so that detector is configured to detect the first and second EMF emissions transmitted through tissue in accordance to a transmittance oximetry measurement scheme;
a housing unit attached to the mouthpiece, wherein the housing unit and the mouthpiece having a commonly shared ventilator passageway;
a blower attached to the housing unit, the blower having an inlet and an outlet, the inlet and the outlet of the blower in fluid communications with the ventilator passageway; and
a control circuit electrically coupled to the first and second EMF emitters, to the detector, and to the blower,
wherein the control circuit is configured to adjust a flow rate of the blower into the ventilator passageway as a function of a detected signal from the detector,
wherein the control circuit is configured to adjust the blower flow rate in response to a detector signal used to estimate an oxygen saturation value in tissue,
wherein the control circuit the control circuit comprises a control unit, a power source, a clock, a visual display, a memory buffer, a data output port, an ON/OFF switch, an AND converter, a first sampler, a second sampler, an amplifier, an EMF drive unit, an alarm unit, a flow rate set unit and a threshold set unit;
inserting the mouthpiece of the device into a mouth of the patient;
switching on the ON/OFF switch to turn on the device;
adjusting manually the flow rate of the blower using the flow rate set unit;
setting a minimum oxygen threshold limit using the threshold set unit;
allowing the patient to sleep with the device secured in the mouth of the patient;
removing the mouthpiece of the device from the mouth of the patient after the patient awakens;
transferring information stored in the memory buffer through the data output port; and
reviewing the transferred information to aid in a sleep apnea diagnosis of the patient.
